(19) United States
(12) Patent Application Publication
(10) Pub. No.: US 2008/0221225 A1
(43) Pub. Date: Sep. 11, 2008

(54) Genes associated with restenosis

(76) Inventors: Euan A. Ashley, Menlo Park, CA (US); Thomas Quertermous, Stanford, CA (US); Eberhard Grube, Siegburg (DE)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/074,964

(22) Filed: Mar. 7, 2008

Related U.S. Application Data
(60) Provisional application No. 60/905,938, filed on Mar. 9, 2007.

(51) Int. Cl. A61K 35/00; C12Q 1/68; A61P 9/00

(52) U.S. Cl. 514/789; 435/6

(57) ABSTRACT

The present invention identifies genes whose gene products are differentially expressed during restenosis, including de novo restenosis and in stent restenosis (ISR). Also provided are therapeutic methods for treating a patient or methods for prophylactically treating an individual susceptible to restenosis. Additionally, the invention describes screening methods for identifying agents that can be administered to treat individuals that have or at risk of developing restenosis.

Since its introduction in 1977, percutaneous coronary intervention (PCI) has revolutionized the treatment of coronary artery disease by providing an alternative to coronary artery bypass graft (CABG) surgery. The subsequent development of coronary stents has had a further positive impact on the clinical effectiveness and predictability of PCI, by significantly reducing peri-procedural abrupt vessel closure and restenosis. However, restenosis, has remained a significant problem.

In-stent restenosis (ISR), although less frequent than post-balloon angioplasty restenosis, is becoming increasingly prevalent as a direct result of the dramatic increase in coronary stent usage. Incidence of ISR in published studies ranges between 10 and 60% and despite early strategies such systemic therapy and direct stenting, only the more recent use of stents coated with anti-proliferative agents has made a significant impact on the disease. Indeed such is the benefit of these ‘drug eluting’ stents that there has been almost universal adoption, at least in the United States, for eligible lesions.

The reduction in luminal diameter following balloon angioplasty is due largely to adventitial constriction with elastic recoil, negative arterial remodelling, thrombus at the injury site, smooth muscle cell proliferation/migration and excessive extracellular matrix production. In contrast, ISR is predominantly a result of neointimal hyperplasia, which, in turn, arises from multiple pathophysiolgies, including thrombus inflammation, as well as intimal and medial dissection. Although a significant relationship between restenosis and long-term mortality has been difficult to assess, nevertheless, when ISR does occur in symptomatic patients, it is difficult to treat and has a high recurrence rate.

Macroscopically, ISR can be classified into either focal or diffuse, with further sub-classifications within each of these two groups. Several studies have consistently identified a number of predictors of both angiographic and clinical restenosis following stent implantation. These include a small (<2.5 mm) reference vessel diameter, occlusions, ostial lesions, lesions within vein grafts, a longer lesion length, use of multiple stents, a history of previous restenosis and the presence of diabetes.

While the scaffolding of a stent controls elastic recoil and negative remodeling, stent-induced vessel injury and inflammatory reactions around stent struts can trigger a set of events that ultimately lead to increased neointimal hyperplasia. Neointimal proliferation, a normal response to vascular damage, is the key mechanism in the progression of in-stent restenosis and the target of the antiproliferative effects of drug-eluting stents.

Drug-eluting stents have been developed to try and minimize in-stent restenosis. However, while sirolimus or paclitaxel eluting stents have consistently demonstrated lower rates of ISR in elective angioplasty of non-bifurcation lesions of mid to large sized arteries in non-diabetics, data outside of these limits is less clear at this time. In addition, concerns over the incidence of late stent thrombosis, related to delayed stent endothelialization, have tempered enthusiasm. Finally, although the occurrence of ISR within drug eluting stents is greatly reduced, it remains around 5%.

For these reasons and others, there is continued interest in potential pharmacotherapeutic agents suitable for stent based delivery to the coronary vasculature. One approach to this is a better understanding of restenosis biology. Classical studies established the central role of platelet-facilitated inflammation following the vascular “injury” of stent deployment. This leads to the migration, de-differentiation and proliferation of smooth muscle cells which then secrete extracellular matrix leading to flow limiting neo-intimal hyperplasia. More in depth characterization is, however, limited by access to human coronary atheromatous material which has resulted in studies with small sample numbers or a focus on small numbers of genes or proteins, by necessity chosen according to a preconceived speculation as to the nature of the disease. More recently, high dimensional genomic and proteomic tools have become available which, when combined with systems biology techniques, allow not just a more comprehensive view of the disease at the transcriptional level, but a view not limited by premeditated stipulation of the process of interest. Moreover, while light microscopy offers an anatomical examination of the cell types involved at the tissue level, molecular profiling can offer insight into the ongoing biological program of those cells. This is especially relevant when the process includes cells which are actively altering their phenotype such as smooth muscle cells.

Recent successes in the abrogation of in stent restenosis (ISR) by drug eluting stents belie the challenges still faced in diabetic patients, complex lesions, and in the occurrence of late stent thrombosis. The present invention addresses the identification of targets of therapy that can avoid these limitations.

### SUMMARY OF THE INVENTION

The present invention provides methods and compositions relating to the analysis of restenosis; and provides molecular targets for development of anti-restenotic agents. A comparative analysis was performed of de novo and in stent restenosis (ISR). Gene ontology analysis demonstrated that cell proliferation programs were prominent in the ISR group and inflammation/immune programs were prominent in the de novo group. The detection of the coding sequence and/or polypeptide products of these genes, as well as molecular pathways in which sets of genes and gene products are involved, provides useful targets for drug screening, as well as providing for early detection, diagnosis, staging, and monitoring of conditions, e.g. by the analysis of blood samples, biopsy material, in vivo imaging, metabolic assays for enzymatic activities, and the like.

The invention provides methods for the identification of compounds that modulate the expression of genes or the activity of gene products in ISR, as well as methods for the treatment of disease by administering such compounds to individuals exhibiting symptoms or tendencies.

The invention also provides a useful model for ISR that allows molecular imaging methods. Such imaging methods are useful in the screening of therapies, e.g. the suppression or activation of genes identified herein, in addition to other genetic, dietary, therapeutic, and other perturbation in lymphatic system. Such screening methods permit evaluation of the efficacy of treatments, and development of novel therapeutics for ISR.

In one embodiment of the invention, the expression profile, or signature profile, of a panel of genes or gene products is evaluated for conditions indicative of various stages of ISR and clinical sequelae thereof. Such a panel may provide a level of discrimination not found with individual markers.

Methods of analysis may include, without limitation, establishing a training dataset, and comparing the unknown sample to the training dataset as test datasets. Alternatively, simple quantitative measure of a panel of genes or gene products may be performed, and compared to a reference to determine differential expression. Other methods may utilize decision tree analysis, classification algorithms, regression analysis, and combinations thereof.

In other embodiments, analysis of differential expression of the above genes or gene products is used in a method of screening biologically active agents for efficacy in the treatment of ISR. In such methods, cells associated with ISR, e.g. smooth muscle cells, endothelial cells, etc., are contacted in culture or in vivo with a candidate agent, and the effect on expression of one or more of the markers, e.g. a panel of markers, is determined. In another embodiment, analysis of differential expression of the above genes or gene products is used in a method of following therapeutic regimens in patients. In a single time point or a time course, measurements of expression of one or more of the markers, e.g. a panel of markers, is determined when a patient has been exposed to a therapy, which may include a drug, combination of drugs, non-pharmacologic intervention, and the like.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Histology of atherectomy specimens. Specimens from de novo atherosclerosis and in stent restenosis are shown. Panels A-B—gross specimens of average and larger size, Panel C—low intima cellularity, Panel D—high intima cellularity, Panel E—an example of a foam cell (arrow), Panel F—lipid deposits (arrow), Panel G—marked inflammatory infiltrate (lympocytes are shown in red), Panel H—a rare example of tunica media.

FIG. 2. Differential gene expression between de novo and in-stent restenosis. Panel A—Graphical representation of false discovery as a function of genes called significant (FDR—false discovery rate). The nadir of the graph represents the optimal balance of true positives to false positives. Panel B—Graphical representation of actual significance (bold black/red line) and mean permuted significance (thin blue line) for this dataset. The further the former departs from the latter, the more significant are the genes. The grey dashed line represents the limit of genes called at a false significance level of 0.77 genes per 207 genes depicted (<1 falsely significant gene, 0.4% false discovery rate, red represents significant genes). All significant genes are higher in the in-stent restenosis group at this level of false discovery. Panel C—heatmap representation of significant genes shown in panel B. Samples are columns and rows are genes. Gene expression is represented as a color, normalized across each row, with brighter red for higher values and brighter green for lower values.

FIG. 3. Ontology analysis of genes differentially expressed between-de novo and in-stent restenosis. Patterns of gene expression in the most upregulated genes in each group were assessed for significant over-representation in the context of molecular function, biological process and cellular component (Gene Ontology Consortium). Panels A-B: genes upregulated in de novo atherosclerosis show a predominantly immune/inflammatory theme. All significant terms are children of the ‘response to stress’ and the ‘response to external stimulus’ parents. Panels C-D: in contrast, the primary molecular signature of in-stent restenosis is cell growth and anion transport.

FIG. 4. Subnets generated using natural language parsing of Medline abstracts were awarded an overall significance score based on the average significance of network members in our experimental data set. Nodes are individual genes while edges are their connecting lines. Each edge represents a relationship revealed by semantic mining. Thicker edges imply a stronger relationship (more associated sentences) Panel A: the ADAM 17 (TNF alpha converting enzyme—TACE) subnetwork. Panel B: the procollagen, type I, alpha 2 gene network. Below each node in the network is a ‘heatstrip’ representation of the raw gene expression data. Brown represents de novo and blue in-stent restenosis. The horizontal center equates to the median expression value for that gene such that the height of vertical bars above or below the line varies according to actual expression values. Nodes are colored according to their significance (bright red—highly significantly upregulated, bright green—highly significantly downregulated).

### DETAILED DESCRIPTION OF THE EMBODIMENTS

The present invention provides methods and compositions relating to the analysis of restenosis; and provides molecular targets for development of anti-restenotic agents. The invention is based, in part, on the evaluation of the expression and role of genes that are differentially expressed in restenotic lesions, and in a comparison of de novo and ISR lesions, including ADAM17 and the ADAM17 pathway, as described herein. Such sequences and signature profiles are useful in the development of drugs; diagnosis; and monitoring of disease. As used herein, gene products may refer to mRNA transcribed from a gene, polypeptides encoded by a gene, or derivatives thereof, e.g. cDNA derived from an mRNA, polypeptide derived from an mRNA, and the like.

To systematically investigate the changes that mediate ISR processes; a comparative analysis was performed of de novo and in stent restenosis (ISR). Gene ontology analysis demonstrated that cell proliferation programs were prominent in the ISR group and inflammation/immune programs were prominent in the de novo group. These findings correlated with results in clinical trials of anti-restenotic agents. Clustering of genes with known functions provides insights into processes and signaling pathways that comprise the development of this disease.

In one embodiment, an investigative platform is provided that defines the proliferative and inflammatory sequences involved in de novo and ISR and provides a relevant basis for future investigation of therapeutic interventions designed to ameliorate the disease and its deleterious effects. In another embodiment, novel pathways that are implicated in the pathogenesis of the disease, allowing assessment of the disease and its responses to therapeutic intervention.

In some embodiments of the invention, the analysis of genetic sequences and protein products thereof in the ADAM17 pathway, including ADAM17 itself, are used in the assessment of restenosis, screening for candidate agents, etc., as described herein. Sequences in this pathway include ADAM17, tumor necrosis factor-alpha converting enzyme; TACE, having the genetic sequence set forth in Genbank accession NM_{—}003183; and as described by Moss et al. (1977) Nature 385:733-736, herein incorporated by reference. One of the most highly differentially regulated subnetworks in the present analysis contained as its nexus ADAM 17, the TNF alpha converting enzyme (TACE). This molecule is believed to regulate inflammation. ADAM17 is shown herein to be highly downregulated in restenotic lesions, as were many members of the subnetwork, suggestive of a role for this module in normal arterial function or in de novo atherosclerosis. These findings argue strongly for TNF system dysregulation in general and ADAM17 in particular as a target for the interruption of atherosclerotic processes.

Analysis of the ADAM17 pathway may further include analysis of ADAM17 in combination with 1, 2, 3, 4, or more sequences in the ADAM17 pathway, which include ADAM9 (Genbank accession NM_{—}003816); ADAM10 (Genbank accession NM_{—}001110); APP (Genbank accession NG_{—}007376); TNFa (Genbank accession NM_{—}000594); TIMP3 (Genbank accession NM_{—}000362); NOS2A (Genbank accession NM_{—}000625); GHR (Genbank accession NM_{—}000163); EGF (Genbank accession NM_{—}001963); TGFa (Genbank accession NM_{—}003236); CD44 (Genbank accession X56794); furin (Genbank accession NM_{—}002569); notch1 (Genbank accession NM_{—}017617); TIMP1 (Genbank accession NM_{—}003254); and TNFrsf1b (Genbank accession NM_{—}001066).

Also of interest is the analysis of genetic sequences and products thereof for nme1 (NM23, nonmetastatic cells 1; nucleoside diphosphate kinase-a; NDPKA). The product of the NM23 gene is a nucleoside diphosphate kinase, which has been designated p19/nm23. The sequences may be accessed at Genbank, NM_{—}198175 and NM_{—}000269, herein specifically incorporated by reference.

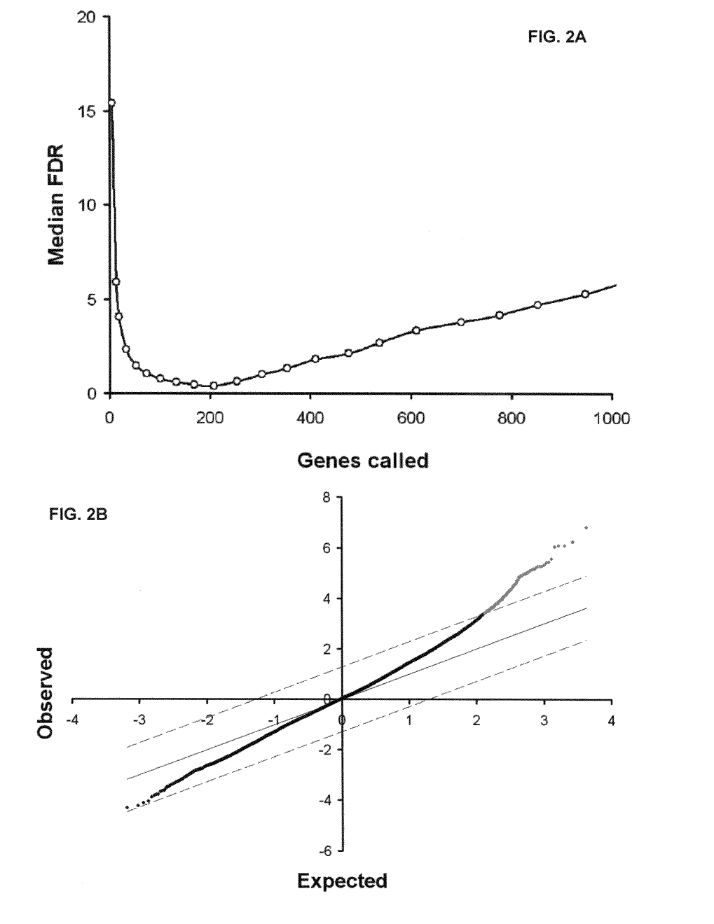

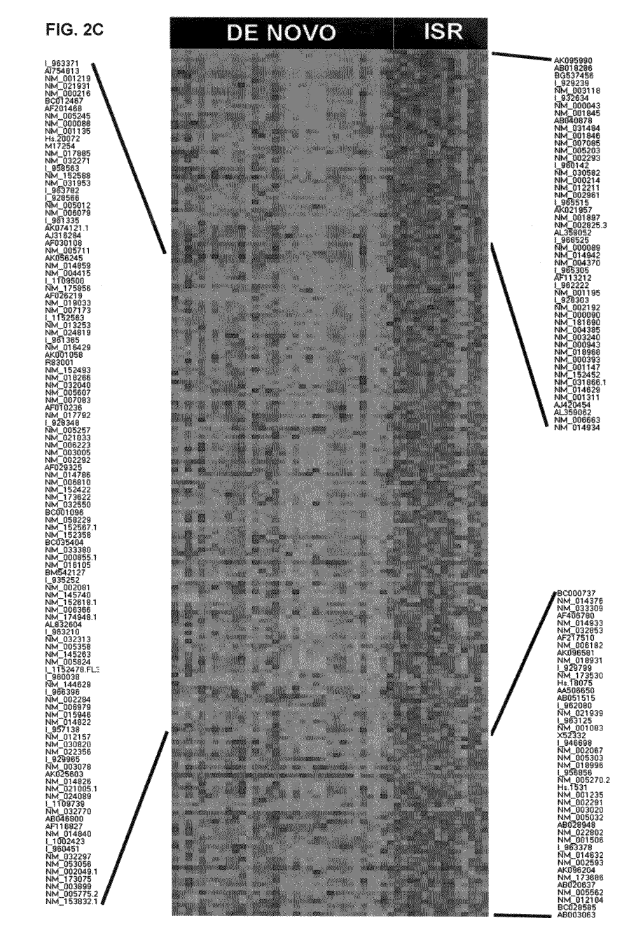

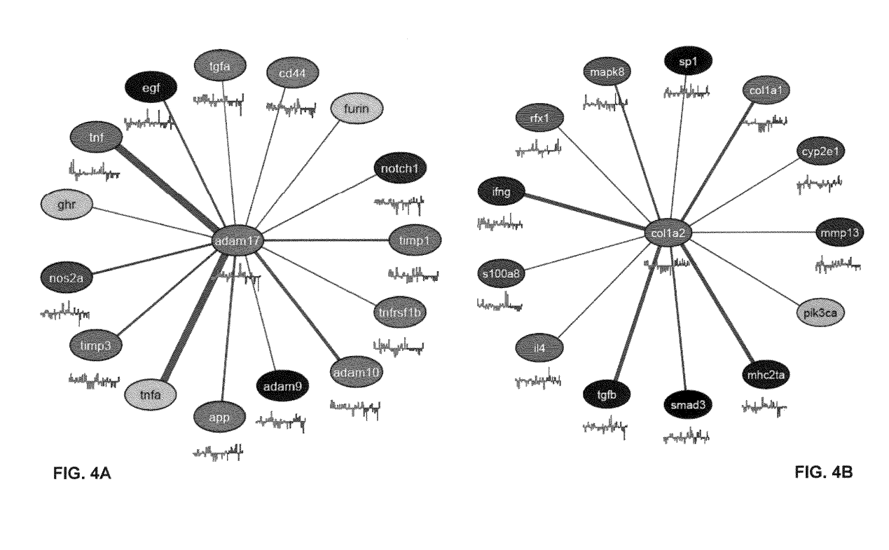

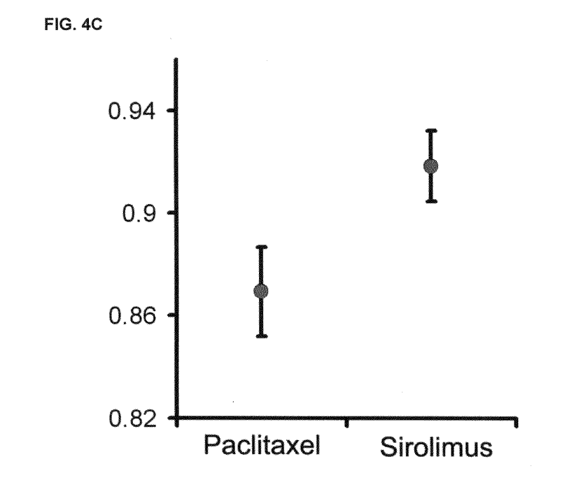

In some embodiments, therapeutic methods are provided for the prevention and/or treatment of ISR with molecular agents that can reduce inflammation, mitigate the immune response, including in particular the molecular pathways shown in FIGS. 4A and 4B, i.e. ADAM17 and molecules involved in the ADAM17 pathway, as shown in FIG. 4A; and col1a2 and molecules involved in the col1a2 pathway. Differentially regulated sequences include those shown in the heat map in FIG. 2C, and the molecules listed in Table 3. Treatments of interest include the coating of stents for prevention of ISR.

As used herein, the term stent is used as is known in the art, to refer to a prosthesis which can be inserted and held, when desired, in a lumen of a vessel or organ in the body. Uses include the support of blood vessels, the trachea, renal and urethral tubules, fallopian tubes, eustachian, large and small intestines, etc. Materials commonly used in stent construction include biologically compatible metals, e.g. stainless steel, titanium, tantalum, gold, platinum, copper and the like, as well as alloys of these metals; low shape memory plastic; a shape-memory plastic or alloy, such as nitinol; and the like. Any of these materials can be fabricated to form channels for use in the present invention, and can form, or be derivatized to form, covalent bonds with the matrix.

Non-limiting examples of commercially available stents include the Gianturco-Roubin stent and the Palmaz-Schatz stent, commonly used for tandem short segment stenotic lesions; Wallstent (Boston Scientific, Natick, Mass.), a self expanding stainless stent used for long lesions; Mammotherm stent, Symphony stent, Smart stent, all of self expanding nitinol; the balloon exapandable Perflex, AVE, Intrastent, and Herculink stents, self-expanding Instent, Gianturco Z-stent (Wilson-Cook, Winston-Salem, N.C.), Ultraflex nitinol mesh stent (Microinvasive, Natick, Mass.), and Esophacoil (IntraTherapeutics, Eden Prairie, Minn.). Tracheobronchial stents include the Gianturco Z tracheobronchial tree stent and the Wallstent tracheobronchial endoprosthesis. The stent may be self-expanding, or may be expandable with a balloon, as is known in the art.

Additional platforms for the invention include polymeric biodegradable stents, anastomotic devices, and scaffolds, including synthetic biodegradable or bioerodible porous scaffolds produced using solid free-form fabrication techniques which include selective laser sintering, three-dimensional printing, fused deposition manufacturing, and stereolithography for micro- or nano-fabrication.

In one embodiment of the invention, the drug or drugs are formulated as a liquid for release from a stent. For example, a stent may include a chamber with a drug transport wall, where the anti-restenotic agent is loaded into the chamber, then selectively transported through the wall (see U.S. Pat. No. 5,498,238). Other variations of this approach include the use of a hollow tubular wire stent, or a stent comprising a reservoir. Such stents are described in the art as having side walls facing outwardly having holes for delivery of the liquid formulation to the targeted site, where the stent is implanted (U.S. Pat. No. 5,891,108). The anti-restenotic agent may be diffused from a reservoir directly to the walls of a blood vessel, through directional delivery openings arranged on an outer surface of the stent. Such devices may also comprise an osmotic engine assembly for controlling the delivery of the agent from the reservoir (U.S. Pat. No. 6,071,305).

An alternative to liquid formulation is provided by devices that comprise a drug compounded to the device itself. In one embodiment, the stent itself is formed of a polymeric material comprising the anti-restenotic agent, where the stent is biodegradable or bioabsorbable (see U.S. Pat. No. 6,004,346). Alternatively, the prostheses may be biostable in which case the drug is diffused out from the biostable materials in which it is incorporated. With metal stents, the device can include a drug-carrying coating overlying at least a portion of the metal.

Alternatively the device may comprise a drug carrying coating. For example a porous stent can be made from a powdered metal or polymer, where the anti-restenotic agents are then compressed into the pores of the stent (see U.S. Pat. Nos. 5,972,027; and 6,273,913). Stents for drug delivery can also comprise multiple coatings, where the rate of release is different for the two coatings (see U.S. Pat. No. 6,258,121), where one of the anti-restenotic agents can be present in both coatings to provide for an extended release profile; or where two or more anti-restenotic agents are differentially released. Other composite coatings include at least one composite layer of the anti-restenotic agent and a polymer material, and at least a barrier layer positioned over the composite layer and being of thickness adequate to provide a controlled release of the bioactive agent (see U.S. Pat. No. 6,335,029). The sheath over the coating containing the anti-restenotic agent can also be perforated, so that when the stent is compressed, the perforation is closed. Upon placement in the vessel, the stent is expanded, and the perforation is opened (see U.S. Pat. No. 6,280,411).

Drugs may be held by covalent bonds (eg, C-C bonds, sulfur bridges) or noncovalent bonds (eg, ionic, hydrogen bonds). The blended matrix may then be attached to the stent surface by dipping or spraying the stent. Drugs may also be released by particle dissolution or diffusion when nonbioerodable matrices are used, or during polymer breakdown when incorporated (absorbed) into a biodegradable substance.

The methods of the invention are practiced by assaying a test agent for its ability to affect a number of biological activities that are positively or negatively correlated with the ability of drugs to inhibit restenosis, in particular by targeting one or more of the pathways and genes identified herein as differentially expressed in restenosis. Functional modulation of ISR associated genes and their products provides a point of intervention to block the pathophysiologic processes leading to disease, and also provides therapeutic intervention. These genes and their products can also be used to prevent, attenuate or reduce damage in prophylactic strategies in patients at high-risk of ISR.

### Identification of Genes Associated with Restenosis

In order to identify restenosis associated genes, lesions were taken from restenotic tissue and evaluated as set forth in the examples. Differentially expressed genes are detected by comparing gene expression levels between the experimental and control conditions. Transcripts within the collected RNA samples that represent differentially expressed genes may be identified by utilizing a variety of methods known to those of skill in the art, including differential screening, subtractive hybridization, differential display, or hybridization to an array comprising a plurality of gene sequences.

“Differential expression” as used herein refers to both quantitative as well as qualitative differences in the genes' temporal and/or tissue expression patterns. Thus, a differentially expressed gene may have its expression activated or inactivated in normal versus disease conditions, or in control versus experimental conditions. Preferably, a regulated gene will exhibit an expression pattern within a given tissue or cell type that is detectable in either control or disease subjects, but is not detectable in both. Detectable, as used herein, refers to an RNA expression pattern or presence of polypeptide product that is detectable via the standard techniques of differential display, reverse transcription- (RT-) PCR and/or Northern analyses, ELISA, RIA, metabolic assays, etc., which are well known to those of skill in the art. Generally, differential expression means that there is at least a 20% change, and in other instances at least a 2-, 3-, 5- or 10-fold difference between disease and control tissue expression. The difference usually is one that is statistically significant, meaning that the probability of the difference occurring by chance (the P-value) is less than some predetermined level (e.g., 5%). Usually the confidence level (P value) is <0.05, more typically <0.01, and in other instances, <0.001.

Table 3 and FIG. 2C provides a list of sequences that have significantly altered expression in restenosis, which genes may be induced or repressed.

### Compound Screening

Compound screening may be performed using polypeptides in vitro, e.g. for binding assays, for functional assays, in cells, including sooth muscle cells, endothelial cells, genetically altered cells or animals, etc. One can identify ligands or substrates that bind to, inhibit, modulate or mimic the action of the encoded polypeptide for a restenosis associated sequence.

The polypeptides include those encoded by the provided genetic sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to a polypeptide encoded by a ISR associated gene, or a homolog thereof.

Compound screening identifies agents that modulate function of the restenosis associated gene or polypeptide. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering or mimicking the physiological function of a restenosis associated gene. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of interfering in a restenosis pathway. The binding assays usually involve contacting a protein with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots. The protein utilized in such assays can be naturally expressed, cloned or synthesized.

Compounds that are initially identified by virtue of acting in a restenosis pathway or by any of the foregoing screening methods can be tested to validate the apparent activity. The basic format of such methods involves administering a lead compound identified during an initial screen to an animal that serves as a model for humans and then determining the effect on, for example, smooth muscle cell proliferation; development of restenosis, etc. The animal models utilized in validation studies generally are mammals. Specific examples of suitable animals include, but are not limited to, primates, rabbits, pigs, mice, and rats.

Active test agents identified by the screening methods described herein can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (Cl) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to modulate gene product activity. Such compounds can then be subjected to further analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

Compounds identified by the screening methods described above and analogs thereof can serve as the active ingredient in pharmaceutical compositions formulated for the treatment of various disorders, including a propensity for restenosis. The compositions can also include various other agents to enhance delivery and efficacy. The compositions can also include various agents to enhance delivery and stability of the active ingredients.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, and intrathecal methods.

Formulations suitable for topical administration, such as, for example, by stent delivery, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Nucleic Acids

The sequences of ISR associated genes find use in diagnostic and prognostic methods, for the recombinant production of the encoded polypeptide, and the like. A list of ISR associated genetic sequences is provided herein. The nucleic acids of the invention include nucleic acids having a high degree of sequence similarity or sequence identity to one of the provided sequences, and also include homologs. Sequence identity can be determined by hybridization under stringent conditions, for example, at 50° C. or higher and 0.1×SSC (9 mM NaCl/0.9 mM Na citrate). Hybridization methods and conditions are well known in the art, see, e.g., U.S. Pat. No. 5,707,829. Nucleic acids that are substantially identical to the provided nucleic acid sequence, e.g. allelic variants, genetically altered versions of the gene, etc., bind to one of the sequences. Further specific guidance regarding the preparation of nucleic acids is provided by Fleury et al. (1997) Nature Genetics 15:269-272; Tartaglia et al., PCT Publication No. WO 96/05861; and Chen et al., PCT Publication No. WO 00/06087, each of which is incorporated herein in its entirety.

The genes listed herein may be obtained using various methods well known to those skilled in the art, including but not limited to the use of appropriate probes to detect the genes within an appropriate cDNA or genomic DNA library, antibody screening of expression libraries to detect cloned DNA fragments with shared structural features, direct chemical synthesis, and amplification protocols. Libraries are preferably prepared from cutaneous cells. Cloning methods are described in Berger and Kimmel, Guide to Molecular Cloning Techniques, Methods in Enzymology, 152, Academic Press, Inc. San Diego, Calif.; Sambrook, et al. (1989) Molecular Cloning—A Laboratory Manual (2nd ed) Vols. 1-3, Cold Spring Harbor Laboratory, Cold Spring Harbor Press, NY; and Current Protocols (1994), a joint venture between Greene Publishing Associates, Inc. and John Wiley and Sons, Inc.

The sequence obtained from clones containing partial coding sequences or non-coding sequences can be used to obtain the entire coding region by using the RACE method (Chenchik et al. (1995) CLONTECHniques (X) 1: 5-8). Oligonucleotides can be designed based on the sequence obtained from the partial clone that can amplify a reverse transcribed mRNA encoding the entire coding sequence. Alternatively, probes can be used to screen cDNA libraries prepared from an appropriate cell or cell line in which the gene is transcribed. Once the target nucleic acid is identified, it can be isolated and cloned using well-known amplification techniques. Such techniques include the polymerase chain reaction (PCR) the ligase chain reaction (LCR), Qβ-replicase amplification, the self-sustained sequence replication system (SSR) and the transcription based amplification system (TAS). Such methods include, those described, for example, in U.S. Pat. No. 4,683,202 to Mullis et al.; PCR Protocols A Guide to Methods and Applications (Innis et al. eds) Academic Press Inc. San Diego, Calif. (1990); Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117.

As an alternative to cloning a nucleic acid, a suitable nucleic acid can be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Left., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences may be cloned and the appropriate subsequences cleaved using appropriate restriction enzymes.

The nucleic acids can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ or 3′ end of the transcribed region. The genomic DNA flanking the coding region, either 3′ or 5′, or internal regulatory sequences as sometimes found in introns, contains sequences required for proper tissue, stage-specific, or disease-state specific expression, and are useful for investigating the up-regulation of expression.

Probes specific to the nucleic acid of the invention can be generated using the nucleic acid sequence disclosed herein. The probes are preferably at least about 18 nt, 25 nt, 50 nt or more of the corresponding contiguous sequence of one of the sequences provided, and are usually less than about 2, 1, or 0.5 kb in length. Preferably, probes are designed based on a contiguous sequence that remains unmasked following application of a masking program for masking low complexity, e.g. BLASTX. Double or single stranded fragments can be obtained from the DNA sequence by chemically synthesizing oligonucleotides in accordance with conventional methods, by restriction enzyme digestion, by PCR amplification, etc. The probes can be labeled, for example, with a radioactive, biotinylated, or fluorescent tag.

The nucleic acids of the subject invention are isolated and obtained in substantial purity, generally as other than an intact chromosome. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other. For hybridization probes, it may be desirable to use nucleic acid analogs, in order to improve the stability and binding affinity. The term “nucleic acid” shall be understood to encompass such analogs.

### Polypeptides

Polypeptides encoded by restenosis associated genes are of interest for screening methods, as reagents to raise antibodies, as therapeutics, and the like. Such polypeptides can be produced through isolation from natural sources, recombinant methods and chemical synthesis. In addition, functionally equivalent polypeptides may find use, where the equivalent polypeptide may be a homolog, e.g. a human homolog, may contain deletions, additions or substitutions of amino acid residues that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. “Functionally equivalent”, as used herein, refers to a protein capable of exhibiting a substantially similar in vivo activity as the polypeptide encoded by an ISR associated gene, as provided in Table II.

Peptide fragments find use in a variety of methods, where fragments are usually at least about 10 amino acids in length, about 20 amino acids in length, about 50 amino acids in length, or longer, up to substantially full length. Fragments of particular interest include fragments comprising an epitope, which can be used to raise specific antibodies. Soluble fragment of cell surface proteins are also of interest, e.g. truncated at transmembrane domains.

The polypeptides may be produced by recombinant DNA technology using techniques well known in the art. Methods that are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized.

### Diagnostic Arrays

Arrays provide a high throughput technique that can assay a large number of polynucleotides or polypeptides in a sample. In one aspect of the invention, an array is constructed comprising one or more probes that specifically bind to restenosis associated genes or gene products, preferably comprising probes specific for at least 5 distinct markers, at least about 10, at least 25, at least 50 or more. This technology is used as a tool to quantitate expression. Arrays can be created by spotting a probe onto a substrate (e.g., glass, nitrocellulose, etc.) in a two-dimensional matrix or array having bound probes. The probes can be bound to the substrate by either covalent bonds or by non-specific interactions, such as hydrophobic interactions. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

The probes utilized in the arrays can be of varying types and can include, for example, antibodies, including antibody fragments or peptidomimetics; synthesized probes of relatively short length (e.g., a 20-mer or a 25-mer), cDNA (full length or fragments of gene), amplified DNA, fragments of DNA (generated by restriction enzymes, for example), reverse transcribed DNA, peptides, proteins, and the like. Arrays can be utilized in detecting differential expression levels.

Common physical substrates for making protein arrays include glass or silicon slides, magnetic particles or other micro beads, functionalized with aldehyde or other chemical groups to help immobilize proteins. The substrate can also be coated with PLL, nitrocellulose, PVDF membranes or modified with specific chemical reagents to adsorb capture agents. The desirable properties of an ideal surface include: chemical stability before, during, and after the coupling procedure, suitability for a wide range of capture agents (e.g., hydrophilic and hydrophobic, low MW and high MW), minimal non-specific binding, low or no intrinsic background in detection, presentation of the capture agents in a fully-functional orientation, production of spots with predictable and regular morphology (shape, signal uniformity).

The variables in the immobilization of proteins include: type of capture agent, nature of surface (including any pretreatment prior to use), and the immobilization method. Both adsorption and covalent attachment have been used for protein arrays. Orientation of the capture agent is very important in presenting it to the ligand or the surface in a functional state. Although covalent attachment using a variety of chemically activated surfaces (e.g., aldehyde, amino, epoxy) as well as attachment by specific biomolecular interactions (e.g., biotin-streptavidin) provide a stable linkage and good reproducibility, chemical derivatization of the surface may alter the biological activity of the capture agent and/or may result in multi-site attachment.

In one embodiment, arrays are made with a non-contact deposition printer. The printer uses thermal ink jet heads that can print many solutions simultaneously to produce hundreds of spots of 50-60 μm diameter with a spacing of 150 μm between spots. The droplet volume ranges between 35 pL to 1.5 nL. The heating element is made out of TaAl or other suitable materials, and is capable of achieving temperatures that can vaporize a sufficient volume of printing buffer to produce a bubble that will push out a precise volume of the antibody solution on the substrate. Selection of printing buffer is important, in that the buffer accomplishes the following: increases printing efficiency (measure of the number of spots that are printed to the total number of spots that are attempted), reduces sample spreading, promotes uniform delivery, stabilizes the capture agents that are being printed, reduces sample drying, increases the visibility of the printed spots. In addition to the printing buffer, other variables that affect printing include: size of the drops, the method of washing and drying the print head, and the speed at which the dispensing head moves. Various modifications may be within these conditions.

Both direct labeling and sandwich format approaches may find use. In the direct labeling procedure, the antibody array is interrogated with serum samples that had been derivatized with a fluorescent label, e.g. Cy3, Cy5 dye, etc. In the sandwich assay procedure, unlabeled serum is first incubated with the array to allow target proteins to be captured by immobilized capture antibodies. Next, the captured target proteins are detected by the application of a labeled detection antibody. The sandwich assay provides extra specificity and sensitivity needed to detect pg/mL concentrations of cytokines, without compromising the binding affinities of the target protein through a direct labeling procedure.

Fluorescence intensity can be determined by, for example, a scanning confocal microscope in photon counting mode. Appropriate scanning devices are described by e.g., U.S. Pat. No. 5,578,832 to Trulson et al., and U.S. Pat. No. 5,631,734 to Stern et al. and are available from Affymetrix, Inc., under the GeneChip™ label. Some types of label provide a signal that can be amplified by enzymatic methods (see Broude, et al., Proc. Natl. Acad. Sci. U.S.A. 91, 3072-3076 (1994)). A variety of other labels are also suitable including, for example, radioisotopes, chromophores, magnetic particles and electron dense particles.

Those locations on the probe array that are bound to sample are detected using a reader, such as described by U.S. Pat. No. 5,143,854, WO 90/15070, and U.S. Pat. No. 5,578,832. For customized arrays, the hybridization pattern can then be analyzed to determine the presence and/or relative amounts or absolute amounts of known species in samples being analyzed as described in e.g., WO 97/10365.

Other methodologies also find use. In some embodiments, a solution based methodology utilizes capillary electrophoresis (CE) and microfluidic CE platforms for detecting and quantitating protein-protein interactions, including antibody reactions with proteins associated with ISR. This technique can be performed easily by any laboratory with access to a standard CE DNA sequencing apparatus. With this methodology, a fluorescent marker (eTag reporter) is targeted to the analyte with one antibody, and a second sandwich antibody of different epitope specificity that is chemically coupled to a “molecular scissors” induces release of the fluorescent probe when both antibodies are in close apposition on the specific analyte. Quantitation then is focused on the liberated eTag, that is quantified with a standard DNA capillary sequencing device. The eTag Assay System can be used to measure the abundance of multiple proteins simultaneously. A critical feature of the assay is that the affinity agents (antibodies) are not immobilized on surfaces, as is required with array technologies. Solution-based binding eliminates surface-induced denaturation and non-specific binding, and improves sensitivity and reaction kinetics.

By combining different colors in the eTag reporters, both mobility and color may be used to dramatically increase the degree of multiplexing. Many binding reactions can be multiplexed in the same vessel, followed by CE to identify the released eTag reporters. Each released eTag reporter encodes the identity of the probe to which it was originally attached. As a result, it is straightforward to configure multiplexed assays to monitor various types of molecular recognition events, especially protein-protein binding.

### Diagnostic Algorithms

An algorithm that combines the results of multiple gene expression level determinations that will discriminate between individuals with restenosis or restenotic tissuese. A statistical test can provide a confidence level for a change in the markers between the test and control profiles to be considered significant. The raw data may be initially analyzed by measuring the values for each marker, usually in triplicate or in multiple triplicates.

A test dataset is considered to be different than the normal control if at least one, usually at least two, at least 5, at least 10 or more of the parameter values of the profile exceeds the limits that correspond to a predefined level of significance.

To provide significance ordering, the false discovery rate (FDR) may be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (see Tusher et al. (2001) PNAS 98, 5116-21, herein incorporated by reference). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The data may be subjected to non-supervised hierarchical clustering to reveal relationships among profiles. For example, hierarchical clustering may be performed, where the Pearson correlation is employed as the clustering metric. One approach is to consider a patient ISR dataset as a “learning sample” in a problem of “supervised learning”. CART is a standard in applications to medicine (Singer (1999) Recursive Partitioning in the Health Sciences, Springer), which may be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

Also provided are databases of expression profiles of restenosis datasets. Such databases will typically comprise expression profiles of individuals having susceptible phenotypes, negative expression profiles, etc., where such profiles are as described above.

The analysis and database storage may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language may be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

The expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

### Diagnostic and Prognostic Methods

The differential expression of restenosis associated genes indicates that these sequences can serve as markers for diagnosis, and in prognostic evaluations to detect individuals at risk for disease, to monitor efficacy of treatment, etc. Prognostic methods can also be utilized to monitor an individual's health status prior to and after an episode, as well as in the assessment of the severity of the episode and the likelihood and extent of recovery.

In general, such diagnostic and prognostic methods involve detecting an altered level of expression of restenosis associated genes or gene products in the cells or tissue of an individual or a sample therefrom, to generate an expression profile. A variety of different assays can be utilized to detect an increase in restenosis associated gene expression, including both methods that detect gene transcript and protein levels. More specifically, the diagnostic and prognostic methods disclosed herein involve obtaining a sample from an individual and determining at least qualitatively, and preferably quantitatively, the level of a restenosis associated genes product expression in the sample. Usually this determined value or test value is compared against some type of reference or baseline value.

The term expression profile is used broadly to include a genomic expression profile, e.g., an expression profile of mRNAs, or a proteomic expression profile, e.g., an expression profile of one or more different proteins. Profiles may be generated by any convenient means for determining differential gene expression between two samples, e.g. quantitative hybridization of mRNA, labeled mRNA, amplified mRNA, cRNA, etc., quantitative PCR, ELISA for protein quantitation, and the like.

The expression profile may be generated from a biological sample using any convenient protocol. While a variety of different manners of generating expression profiles are known, such as those employed in the field of differential gene expression analysis, one representative and convenient type of protocol for generating expression profiles is array based gene expression profile generation protocols. Following obtainment of the expression profile from the sample being assayed, the expression profile is compared with a reference or control profile to make a diagnosis regarding the susceptibility phenotype of the cell or tissue from which the sample was obtained/derived. Typically a comparison is made with a set of cells from an unaffected, normal source. Additionally, a reference or control profile may be a profile that is obtained from a cell/tissue known to be predisposed to restenosis, and therefore may be a positive reference or control profile.

In certain embodiments, the obtained expression profile is compared to a single reference/control profile to obtain information regarding the phenotype of the cell/tissue being assayed. In yet other embodiments, the obtained expression profile is compared to two or more different reference/control profiles to obtain more in depth information regarding the phenotype of the assayed cell/tissue. For example, the obtained expression profile may be compared to a positive and negative reference profile to obtain confirmed information regarding whether the cell/tissue has the phenotype of interest.

The difference values, i.e. the difference in expression in the presence and absence of radiation may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above. A statistical analysis step is then performed to obtain the weighted contribution of the set of predictive genes.

In one embodiment of the invention, blood samples, or samples derived from blood, e.g. plasma, serum, etc. are assayed for the presence of polypeptides encoded by restenosis associated genes, e.g. cell surface and, of particular interest, secreted polypeptides. Such polypeptides may be detected through specific binding members. The use of antibodies for this purpose is of particular interest. Various formats find use for such assays, including antibody arrays; ELISA and RIA formats; binding of labeled antibodies in suspension/solution and detection by flow cytometry, mass spectroscopy, and the like. Detection may utilize one or a panel of specific binding members, e.g. specific for at least about 2, at least about 5, at least about 10, at least about 15 or more different gene products. A subset of genes and gene products of interest for assays are provided in Table II.

In another embodiment, in vivo imaging is utilized to detect the presence of restenosis associated gene. Such methods may utilize, for example, labeled antibodies or ligands specific for cell surface restenosis associated gene products. Included for such methods are gene products differentially expressed in lesion samples, which can be localized by in situ binding of a labeled reagent. In these embodiments, a detectably-labeled moiety, e.g., an antibody, ligand, etc., which is specific for a polypeptide of interest is administered to an individual (e.g., by injection), and labeled cells are located using standard imaging techniques, including, but not limited to, magnetic resonance imaging, computed tomography scanning, and the like. Detection may utilize one or a cocktail of imaging reagents e.g. imaging reagents specific for at least about 2, at least about 5, at least about 10, at least about 15 or more different gene products.

In another embodiment, an mRNA sample from cutaneous tissue is analyzed for the genetic signature indicating restenosis. Expression signatures typically utilize a panel of genetic sequences, e.g. a microarray format; multiplex amplification, etc., coupled with analysis of the results to determine if there is a statistically significant match with a disease signature.

Nucleic acids or binding members such as antibodies that are specific for polypeptides derived from the sequence of one of the sequences provided can be used to screen patient samples for increased expression of the corresponding mRNA or protein. Samples can be obtained from a variety of sources. For example, since the methods are designed primarily to diagnosis and assess risk factors for humans, samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from whole skin, dermal, layers, epidermal layers, etc. Also included in the term are derivatives and fractions of such cells and fluids. Where cells are analyzed, the number of cells in a sample will often be at least about 10², usually at least 10³, and may be about 10⁴ or more. The cells may be dissociated, in the case of solid tissues, or tissue sections may be analyzed. Alternatively a lysate of the cells may be prepared.

Diagnostic samples are collected any time after an individual is suspected to have restenosis, etc. or has undergone an event that predisposes to restenosis. In prophylactic testing, samples can be obtained from an individual who present with risk factors that indicate a susceptibility, which risk factors include breast cancer surgery, etc. as part of a routine assessment of the individual's health status.

The various test values determined for a sample from an individual believed to suffer restenosis, and/or a tendency to restenosis typically are compared against a baseline value to assess the extent of increased or decreased expression, if any. This baseline value can be any of a number of different values. In some instances, the baseline value is a value established in a trial using a healthy cell or tissue sample that is run in parallel with the test sample. Alternatively, the baseline value can be a statistical value (e.g., a mean or average) established from a population of control cells or individuals. For example, the baseline value can be a value or range that is characteristic of a control individual or control population. For instance, the baseline value can be a statistical value or range that is reflective of expression levels for the general population, or more specifically, healthy individuals not susceptible to restenosis.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE 1

We recruited 102 patients who underwent coronary atherectomy for de novo atherosclerosis or ISR. Samples were fixed for histology and near genome-wide gene expression was assessed using a dual dye 22 k microarray. Histological analysis revealed significantly greater cellularity and significantly fewer inflammatory infiltrates and lipid pools in the ISR group. Gene ontology analysis demonstrated that cell proliferation programs were prominent in the ISR group and inflammation/immune programs were prominent in the de novo group. Network analysis, which combines semantic mining of the published literature with the expression signature of ISR, revealed gene expression modules that are suggested as candidates for selective abrogation of restenotic disease. Two modules are presented in more detail, the procollagen type 1 alpha 2 gene (COL1A2) and the ADAM17/TNF alpha converting enzyme gene. Finally, we tested our contention that this method is capable of identifying successful targets of therapy by comparing mean significance scores for networks generated from subsets of the published literature containing the terms ‘sirolimus’ or ‘paclitaxel’. In addition, we generated two large networks with sirolimus and paclitaxel at their centers. Both analyses revealed higher mean values for sirolimus suggesting, in line with results from recently published clinical trials, that this agent has a broader suppressive action vs ISR than paclitaxel. Comprehensive histological and gene network analysis of human ISR reveals potential targets for directed abrogation of restenotic disease and recapitulates the results of major clinical trials of existing agents.

### Materials and Methods

Development of oligonucleotide microarray platform. A custom microarray was developed as described in detail in King et al. (2005) Physiol Genomics 23:103-18 2005. In brief, a combination of techniques based on experimental cell culture and data mining was used to provide a comprehensive catalog of vascular and atherosclerosis related genes. Probes derived from this list were combined with those from Agilent human catalog arrays 1A and 1B to create the final 22 k feature microarray.

Patient recruitment and tissue harvest. One hundred and two patients with lesions suitable for coronary atherectomy were recruited at the Siegburg Heart Center (Siegburg, Germany). Standard premedication with oral aspirin, clopidogrel and intracoronary heparin was given and femoral arterial access was gained using 6 French or 8 French introducers. Following diagnostic angiography, longitudinal atherectomy was performed using the Silverhawk atherectomy catheter (Foxhollow Technologies, Redwood City, Calif.) facilitated by the use of an extra support guidewire. The total number of cuts applied to reduce the plaque load was at the operator's discretion. Extracted specimens were divided and stored immediately in both liquid nitrogen and formalin. There were 89 samples with integrity adequate for histological analysis. Seventy samples had matching RNA, histology and clinical information. Of these, fifty six samples had RNA of sufficient quality for hybridization and forty seven arrays were judged high enough quality for analysis after rigorous quality control.

Histology. Tissue samples were fixed in 10% neutral buffered formation, processed routinely through graded alcohols and clearing agent, and embedded in paraffin blocks. Tissue sections (5 μm thick) were cut, and stained with hematoxylin and eosin, Gomori's Trichrome, and elastic Van Gieson stain. After exclusion of 13 samples from chronic total occlusion lesion, the sections were classified as de novo atherosclerosis or ISR and evaluated microscopically for intimal cellularity, lipid deposit, inflammatory cell infiltration, thrombosis, and calcification.

RNA isolation and quantification. RNA was isolated using standard techniques based on guanidium thiocyanate as described previously. Extensive quality control testing of all RNA was carried out. Samples were quantified with the NanoDrop ND-1000 Spectrophotometer (NanoDrop, Wilmington, Del.) and RNA integrity assessed using the 2100 Bioanalyzer System and RNA 6000 Pico LabChip Kit (Agilent Technologies, Palo Alto, Calif.).

Direct labeling and oligonucleotide array hybridization. Our protocol has been described in detail previously. In brief, 10 micrograms of RNA for sample and reference were used. A mixture of 80% HeLa RNA and 20% human umbilical vein endothelial cell RNA was used for reference as this had previously been shown to maximize feature fluorescence for this array. Fluorescent labeled cDNA was reverse transcribed from the RNA using SuperScript II (Invitrogen) and either cyanine 3-dCTP or cyanine 5-dCTP (Perkin-Elmer/NEN, Boston, Mass.). After purification, hybridization continued for 16 hours at 60° C. Arrays were scanned using the G2565AA Microarray Scanner System and features extracted as previously described. Feature values excluded according to the above criteria were estimated using the KNN imputation method of Troyanskaya et al. (2001) Bioinformatics. 17:520-5.

Analytical methods. Proportions of categorical variables in the histology analysis were compared using Fisher's exact test. Our primary difference measure for genomic analysis was the Significance Analysis of Microarrays. Tusher et al. (2001) Proc Natl Acad Sci USA. 98:5116-21. Here a significance score is calculated (□ₖ₍ᵢ₎−|ⱼ₍ᵢ₎/sᵢ+s₀ where □ₖ=mean expression of gene k in ISR group, yⱼ=mean expression of gene k in de novo atherosclerosis group, Si is the standard deviation of repeated expression measurements, s₀ is a small positive constant and k=gene of interest). The metric is a modified t statistic (labeled ‘d’ because of the addition of a small positive constant to the denominator). Multiple testing is accounted for by bootstrap permutation. Results from difference analysis are displayed using Heatmap software.

Differentially expressed genes were analyzed in the context of Gene Ontology (GO) in order to identify groups with similar functions or processes. In brief, all genes represented on the microarray were assigned to corresponding GO terms using the Biomolecule Naming Service (BNS). A gene was associated with a GO term if it was annotated by this term or its child. For each GO term, we computed a p-value based on the hypergeometric distribution by comparing the number of genes annotated by the GO term in a given list of differentially expressed genes, with the expected number of such genes.

We have previously described a method for identifying highly ‘connected’ genes by semantic mining of the published literature. We termed these genes ‘nexus’ genes to emphasize their central role in biological networks and to make a distinction with the ‘hub’ gene concept, where connections are derived from a form of network analysis centered around correlation of gene expression. These concepts share the implication that biological pathways contain ‘hot spots’ where therapeutic targeting will lead to disproportionate biological effects. It is our contention that the identification and targeting of pathways at these points will lead to significantly greater therapeutic benefit than targeting elsewhere. In our method, we use semantic mining of the published literature. An association network is derived from language parsing of Medline abstracts and association identified between any two genes if they sandwich, in the same sentence, an interaction verb as defined by our user context file. A series of subnetworks (independent of our experimental data) is then generated and the expression values and significance of genes in our analysis ‘overlaid’ visually and mathematically on these networks. An overall significance score for each subnetwork is calculated by using the mean d score value for all members or the cumulative sum of d score values for all members. Higher scores thus indicate greater significance. The method has been described in detail previously.

To test our hypothesis that connectivity analysis is capable of identifying targets which may lead to more efficacious therapies, we took advantage of the recent publication of multiple head to head trials of paclitaxel and sirolimus eluting stents. These clinical trials demonstrated a small but consistent and significant benefit of sirolimus over paclitaxel in the abrogation of ISR. We posited that our methods should be capable of predicting this result. Thus, two abstract sub-databases were generated by entering the following queries into Medline “(rapamycin OR sirolimus) NOT paclitaxel” and “paclitaxel NOT sirolimus NOT rapamycin”. Network analysis was then carried out within these abstract databases and the overall significance of each subnetwork calculated as detailed above. The means for sirolimus and paclitaxel groups were compared using Student's t test. In a separate analysis we incorporated the terms “sirolimus” and “paclitaxel” directly as interacting gene ‘terms’ to generate two large additional networks. Overall significance was also compared for these networks.

Pathway interactions are mapped using Cytoscape (version 2.2) (Shannon et al. (2003) Genome Res. 13:2498-504) with the Agilent literature search plugin (v. 2). Using a separate routine, significance levels are overlaid as colors and raw data depicted as a short ‘heatstrip’ below each node.

### Results

Patient population. Patient characteristics corresponding to the 47 samples in the genomic analysis are detailed in Table 1.

TABLE 1

| | De Novo | In stent restenosis |
|---|---|---|
| n | 32 | 15 |
| Age, yr | 66 (±11.85) | 70 (±7.66) |
| Male | 21 (84%) | 9 (64.28%) |
| unknown | | 1 (7.14%) |
| Ethnicity | | |
| caucasian | 21 (84%) | 14 (100%) |
| asian | 1 (4%) | |
| unknown | 3 (12%) | |
| Hypertension | | |
| yes | 13 (52%) | 10 (71.42%) |
| no | 7 (28%) | 2 (14.28%) |
| unknown | 5 (20%) | 2 (14.28%) |
| Hypercholesterolemia | | |
| yes | 15 (60%) | 12 (85.71%) |
| no | 6 (24%) | 2 (14.28%) |
| unknown | 4 (16%) | |
| Diabetes | | |
| yes | 1 (4%) | 2 (14.28%) |
| no | 11 (44%) | 8 (57.14%) |
| unknown | 13 (52%) | 4 (28.57%) |
| Tobacco | | |
| yes | 5 (20%) | 4 (28.57%) |
| no | 20 (80%) | 10 (71.42%) |
| Previous ACS | | |
| yes | 11 (44%) | 12 (85.71%) |
| no | 4 (16%) | |
| unknown | 10 (40%) | 2 (14.28%) |
| Drug therapy | | |
| ACE inhibitors | 10 (40%) | 7 (50%) |
| ARB | 0 | 0 |
| B-blockers | 15 (60%) | 9 (64.28%) |
| Ca-channel | 3 (12%) | 2 (14.28%) |
| antagonist | | |
| Statin | 14 (56%) | 11 (78.57%) |

Patient characteristics for the 47 samples included in the genomic analysis.
Age is presented ± standard deviation.

TABLE 2

| | De Novo | ISR | |
|---|---|---|---|
| Lesions | % (cases) | % (cases) | P |
| Low cellularity | 45 (81.8%) | 9 (26.5%) | <0.0001 |
| High cellularity | 10 (18.2%) | 25 (73.5%) | <0.0001 |
| Lipid deposit | 14 (25.5%) | 1 (2.9%) | 0.001 |
| Inflammatory | 21 (38.2%) | 3 (8.8%) | 0.0004 |
| focus | | | |
| Thrombus | 11 (20%) | 4 (11.8%) | 0.25 |
| Calcification | 3 (5.5%) | 1 (2.9%) | 0.66 |
| Number of cases | 55 | 34 | |

Histological features of 55 de novo and 34 in stent restenosis lesions. Cellularity was significantly higher in the in stent restenosis group while lipid deposits and inflammatory foci were more common in the de novo group. There was no difference in the frequency of thrombus or calcification.

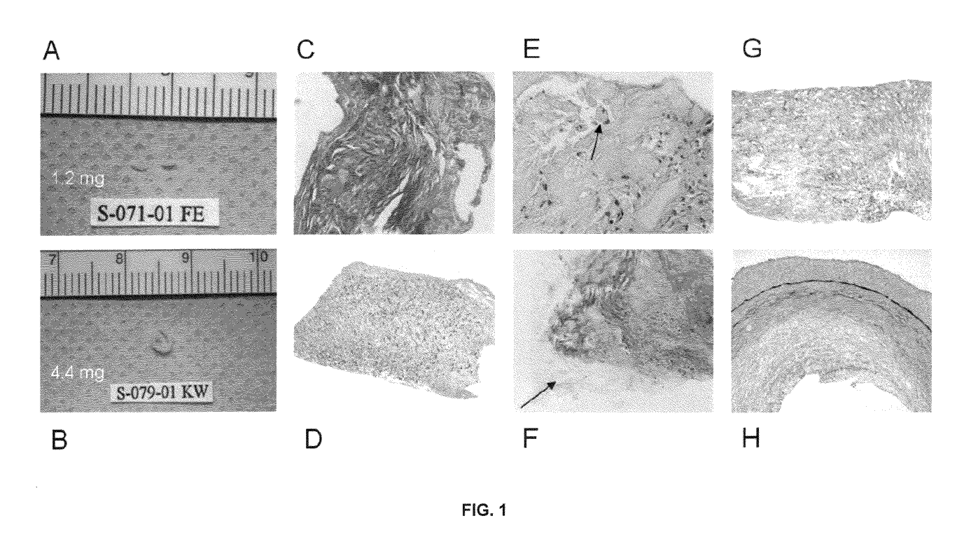

Gross specimen and histological analysis. Lesions were classified as ‘de novo’ or ‘in-stent restenosis’ by the operator at the time of catheterization. Samples were weighed before embedding and fixation (mean 1.22±1.02 mg, range 0.1 to 4.77 mg, FIG. 1 Panels A-B). Histological analysis (FIG. 1, Panels C-H, Table 1) revealed significantly greater frequency of “low cellularity” lesions (Panel C) in the de novo group while there was a corresponding higher frequency of “high cellularity” lesions (Panel D, both p<0.0001) within the intima of ISR lesions. De novo lesions exhibited classically recognized features of atherosclerosis were found more frequently in de novo lesions than in restenosis lesions: foam cells (Panel E), lipid pools (Panel F, p=0.001) and inflammatory infiltrates (Panel G, p=0.0004). There was no difference in the incidence of calcification or thrombosis between groups.

Gene expression analysis. Comparison analysis using the significance analysis of microarrays revealed highly significant differences between de novo lesions and ISR lesions. Plotting the false discovery characteristics (FIG. 2, Panels A-B) reveals a nadir rate of false discovery at around 200 genes (0.77 genes per 207 genes depicted, <1 falsely significant gene, 0.4% false discovery rate). In addition, at this level of false discovery, all significant genes had greater expression in the in-stent restenosis group (FIG. 2, Panels B-C).

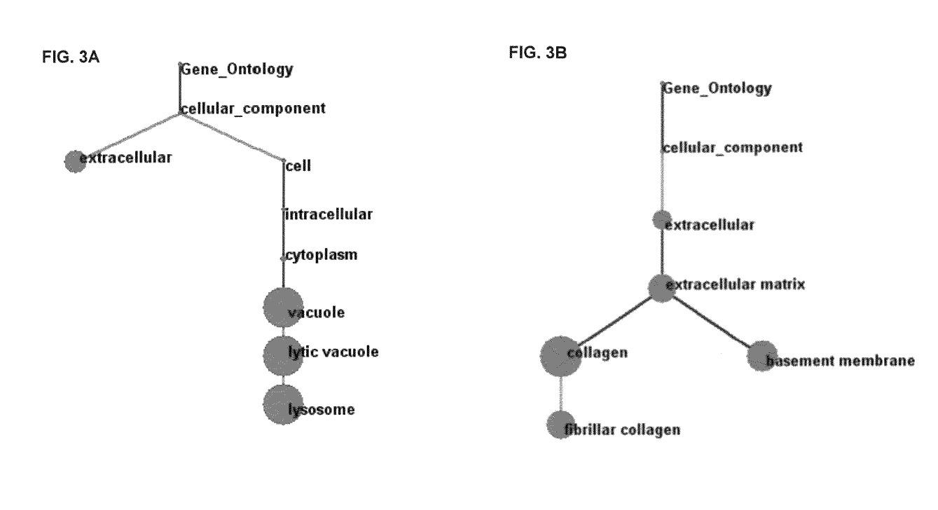

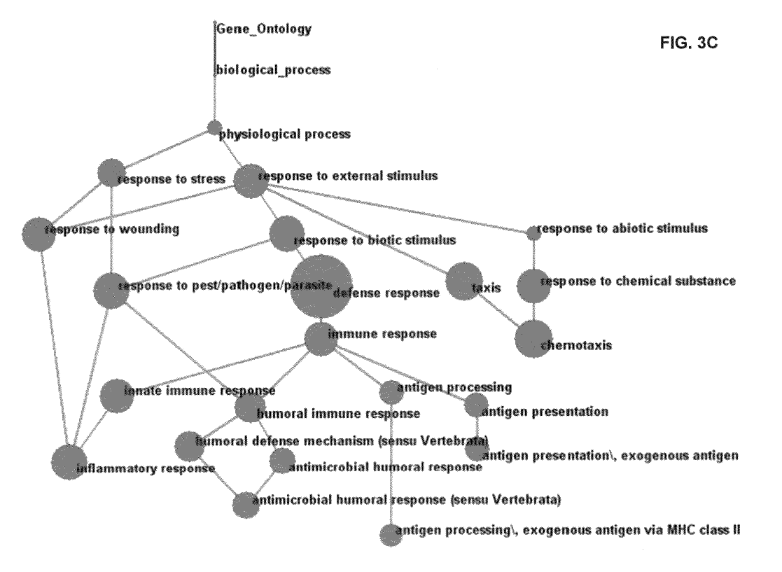

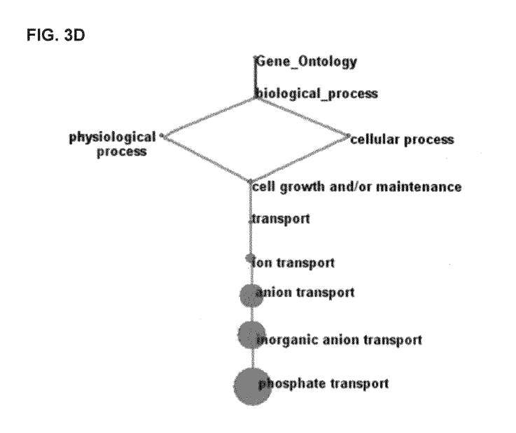

Ontology analysis. While gene lists are informative, systems analysis lends structure to large data. Patterns of gene expression within the most upregulated genes in each group were assessed for over-representation in the context of molecular function, biological process and cellular component (Gene Ontology Consortium). Significant ontologies are depicted in FIG. 3. As shown, the primary molecular signature differentiating de novo atherosclerosis from in-stent restenosis is an immune/inflammatory one (FIG. 3, Panels A-B). All significant terms in genes upregulated in de novo atherosclerosis are children of the ‘response to stress’ and the ‘response to external stimulus’ parents. This is an important finding because ontology analysis is ‘blind’ to prespecified pathways of interest. In contrast, the primary molecular signature of in-stent restenosis is cell growth and anion transport (Panels C-D). While previous work suggests inflammation is an important part of early stage ISR, we found no evidence of overrepresentation of inflammatory ontologies compared to de novo atherosclerosis in our data.

Network analysis: We generated subnets using natural language parsing of Medline abstracts and awarded each an overall significance score based on the average significance of network members within our experimental data set (Table 3). Subnetworks were explored using Cytoscape and sentences curated for the most promising candidate networks. Example subnetworks are reported in Table 3 and illustrated in FIG. 4. The analysis provided a highly enriched source of candidates, both expected (for example, collagen genes, fibroblast receptor gene, interleukin 8) and unexpected (ADAM genes, nm23 protein gene). FIG. 4 shows the ADAM 17 subnetwork. ADAM 17 is the TNF alpha converting enzyme (TACE) and it is believed to regulate inflammation. Other notable members of the ADAM17 subnetwork include other disintegrin and metalloproteinase domain (ADAM) proteins, inducible nitric oxide synthase, tissue inhibitors of matrix metalloproteinases and EGF.

TABLE 3

| | | | d | Cumu- | |
|---|---|---|---|---|---|
| Symbol | Name | Nodes | score | lative | Mean |
| col5a2 | collagen, type V, alpha 2 | 7 | 4.62 | 17.38 | 2.48 |
| col1a1 | collagen, type I, alpha 1 | 10 | 4.32 | 20.45 | 2.04 |
| adam10 | a disintegrin and | 11 | −2.72 | 22.22 | 2.02 |
| | metalloproteinase domain 10 | | | | |
| map4k4 | MAP kinase kinase kinase | 10 | 3.91 | 19.66 | 1.97 |
| | kinase 4 | | | | |
| col3a1 | collagen, type III, alpha 1 | 7 | 4.86 | 13.29 | 1.90 |
| col1a2 | collagen, type I, alpha 2 | 14 | 4.97 | 29.75 | 1.86 |
| gli3 | GLI-Kruppel family member | 9 | 2.52 | 15.67 | 1.74 |
| | GLI3 | | | | |
| gpx1 | glutathione peroxidase 1 | 13 | −3.20 | 22.37 | 1.72 |
| il8ra | interleukin 8 receptor, alpha | 9 | −0.38 | 15.13 | 1.68 |
| serpinf1 | serine (or cysteine) proteinase | 13 | −2.37 | 21.49 | 1.65 |
| | inhibitor, clade F member 1 | | | | |
| adam17 | a disintegrin and | 15 | −2.46 | 30.78 | 1.62 |
| | metalloproteinase domain 17 | | | | |
| | (tumor necrosis factor, alpha, | | | | |
| | converting enzyme) | | | | |
| nme1 | non-metastatic cells 1, protein | 17 | 0.83 | 27.26 | 1.60 |
| | (NM23A) | | | | |
| fgfr2 | fibroblast growth factor | 9 | 0.09 | 14.18 | 1.58 |
| | receptor 2 | | | | |

Selected subnetworks ranked according to the mean significance score of the network.
Nodes is the number of nodes in each subnetwork;
d score is the significance level of the nexus gene (the higher the positive number the more significant the upregulation, the lower the negative number the more significant the downregulation);
Cumulative is the summed significance value for the whole network;
Mean is cumulative divided by number of nodes.
The list is highly enriched for collagen genes, and inflammatory genes such as ADAM proteins and interleukin 8 receptor alpha.

Targets for inhibiting in-stent restenosis include differentially regulated networks with nexus genes highly upregulated in this tissue. Several are reported in Table 3 and one is shown in FIG. 4, Panel B. The procollagen, type I, alpha 2 gene (COL1a2) was as highly upregulated as any nexus gene in the top 50 subnetworks (d score 4.97). The subnetwork contained other collagen genes (COL1A1), regulatory elements (Sp1) as well as key players in inflammation: interferon gamma (IFNG), TGF beta, major histocompatibility class II transactivator (CIITA), interleukin 4 (IL4), fundamental signaling molecules (PI3 kinase, MAP kinase 8), and matrix metalloproteinase 13 (MMP13).

To test the idea that useful targets of therapy could be identified by our approach, we took advantage of recently published head to head clinical trials of two agents effective against ISR. Networks were generated using subsets of the published literature which contained the terms “sirolimus or rapamycin NOT paclitaxel”, or the reverse as noted above. The paclitaxel search generated 11,136 abstracts while the sirolimus search generated 4,854 abstracts. Despite fewer abstracts, the sirolimus network contained more gene associations. The sirolimus network consisted of 818 nodes and 3,690 associations while the paclitaxel network consisted of 611 nodes and 1,862 associations. The overall significance levels of subnets with greater or equal to five nodes are shown in FIG. 4, Panel C. Empiric testing suggests average d scores for subnetworks with less than 5 nodes are disproportionately affected by one or two very significant interactions. We hypothesized that whichever of the agents was more effective should demonstrate a higher mean significance level amongst its subnetworks. As shown, the mean significance level of subnetworks in the sirolimus groups was significantly higher than for the paclitaxel group (p=0.028) suggesting that abstracts which included sirolimus in their content were likely to contain genes more significantly different between de novo and ISR than those which included paclitaxel. Further, when individual networks were generated by incorporating the terms ‘sirolimus’ and ‘paclitaxel’ directly as interacting nodes, the sirolimus network (518 associations) had an overall significance level (0.85) which was higher than that of paclitaxel (294 associations, overall significance level 0.81). Together, these results support the idea that sirolimus has a broader efficacy of action in targeting genetic modules significantly different between de novo and ISR groups.

### Discussion

We present here a comprehensive biological characterization of ISR. In addition to light microscopy, we have used high dimensional genomic tools to characterize the transcriptional signature of ISR and, by subtracting the molecular profile of de novo atherosclerosis, we have applied systems analysis to make predictions of potential new therapeutic targets. Finally, we have tested our method against recently published clinical trials and find that our analysis supports the previously demonstrated broader efficacy of sirolimus over paclitaxel in the abrogation of in stent restenotic disease.

In fact, surprisingly few studies have examined the basic cellular processes of in stent restensosis, presumably because tissue is hard to acquire. Many studies have examined circulating markers or predictors and a small number of studies have begun to describe allelic variation associated with the restenotic response. Indeed, a large NIH study is currently underway to comprehensively assess the genomics of ISR. In our study, we confirmed findings from cellular microscopy: ISR consists primarily of smooth muscle cells, seen in much greater number than in de novo atherosclerosis, and proteoglycan matrix. Occasional focal collections of inflammatory cells and lipid pools are seen but these are more common in de novo disease while thrombus and calcification occur at the same frequency in each group. While revealing, cellular anatomy offers only limited insight into cellular function.

Our technique of using gene expression profiles and network analysis is designed to enrich for genetic modules of interest. One of the most highly differentially regulated subnetworks in our analysis contained as its nexus ADAM 17, the TNF alpha converting enzyme (TACE). This molecule is believed to regulate inflammation via cleavage and release of transmembrane proteins from the cell surface including TNF alpha and its receptors TNFR1 and TNFR2. As such, potentially, both pro and anti-inflammatory effects could result: release of soluble TNF would enhance inflammation while release of a TNF receptor ectodomain might limit the effects of soluble TNF. In our analysis, TACE itself was highly downregulated (d score −2.46) as were many members of the subnetwork, suggestive of a role for this module in normal arterial function or in de novo atherosclerosis. These findings argue strongly for TNF system dysregulation in general and ADAM17 in particular as a target for the interruption of atherosclerotic processes.

Highly upregulated nexus genes in significantly differentially regulated networks are attractive targets for the abrogation of ISR. The procollagen type 1 alpha 2 subnetwork (COL1A2) illustrated in FIG. 4 is one such example and one which suggests potential targets for its own suppression. For example, decoy Sp1 binding oligonucleotides have been shown to inhibit COL1A2 promoter activity both in cultured fibroblasts and in vivo, in the skin of transgenic mice. Alternatively, interferon gamma (IFNG) represses collagen in a manner requiring a class 11 transactivator molecule (CIITA). Both of these molecules are significantly downregulated in the COL1A2 subnetwork. Thus, a specific hypothesis generated by our data is that targeting of COL1A2 would abrogate ISR. Further, we contend that this directed approach is more elegant than the general inhibition of cell proliferation of sirolimus, paclitaxel or brachytherapy, and thus is likely to result in less unwanted effects such as slow endothelialization and late stent thrombosis.

We have previously shown that high dimensional techniques can identify targets for treatment in heart failure. To test the idea that such targets in vascular biology could be identified by our approach, we took advantage of recently published head to head clinical trials of two agents effective against ISR. Although differences in late lumen loss or target lesion revascularization in these trials could be explained by factors relating to deployment, stent design, polymer design, agent or time course of elution, we hypothesized that the principal factor was the agent itself. We found that in networks generated from subsets of the published literature focused on sirolimus or paclitaxel, the mean significance level of subnetworks in the sirolimus group was greater than for the paclitaxel group. Thus our method predicts the results of multiple large scale clinical trials.

In conclusion, we have carried out a comprehensive assessment of the biology of human ISR. We confirmed previously reported findings from cellular microscopy studies and extended these into the molecular domain with near genome-wide transcriptional profiling. Ontology analysis of differentially regulated genes revealed a significantly more inflammatory profile for de novo atherosclerosis lesions than ISR lesions. Network analysis confirmed this finding and identified subnetworks of highly connected genes which we hypothesize to be promising therapeutic targets. Finally, we tested our method by forcing it to predict which of sirolimus or paclitaxel would be the more effective agent in abrogating ISR and found that it successfully recapitulated the results of large scale clinical trials. This is the first implementation of a network based analysis of ISR and represents the most comprehensive survey of the disease process to date.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

What is claimed is:

1. A method for developing biologically active agents that modulate activity of a restenosis target gene or gene product, the method comprising:
combining a candidate biologically active agent with any one of:
(a) a restenosis target polypeptide;
(b) a cell comprising a nucleic acid encoding and expressing a restenosis associated polypeptide; and
determining the effect of said agent on restenosis associated molecular and cellular changes.

2. The method according to claim 1, wherein said biologically active agent downregulates or upregulates expression.

3. The method according to claim 1, wherein said biologically active agent inhibits or increases activity of said polypeptide.

4. The method of claim 1, wherein the restenosis target peptide is a member of the ADAM17 pathway.

5. The method of claim 4, wherein the member of the ADAM 17 pathway is selected from ADAM17; ADAM9; ADAM10; APP; TNFa; TIMP3; NOS2A; GHR; EGF; TGFa; CD44; furin; notch1; TIMP1; and TNFrsf1b.

6. The method of claim 5, wherein the restenosis target peptide is ADAM17.

7. A method for the diagnosis or staging of a restenotic lesion, the method comprising:
determining the upregulation or downregulation of expression of a restenosis associated gene or polypeptide.

8. The method according to claim 7, wherein said determining comprises detecting increased or decreased amounts of mRNA or polypeptide in intimal lesion cells.

9. The method of claim 8, wherein the restenosis associated gene or polypeptide is a member of the ADAM17 pathway.

10. The method of claim 9, wherein the member of the ADAM 17 pathway is selected from ADAM17; ADAM9; ADAM10; APP; TNFa; TIMP3; NOS2A; GHR; EGF; TGFa; CD44; furin; notch1; TIMP1; and TNFrsf1b.

11. The method of claim 10, wherein the restenosis target peptide is ADAM17.

12. A method to treat restenosis, the method comprising:
administering a therapeutic amount of a compound identified by the method according to claim 1.
